(19) United States
(12) Patent Application Publication
(10) Pub. No.: US 2004/0171952 A1
(43) Pub. Date: Sep. 2, 2004

(54) Mechanism of wireless heartbeat transmitter

(75) Inventor: Shui Jung Chen, San Chung City (TW)

Correspondence Address: CHAUNCEY B. JOHNSON, JOHNSON & ASSOCIATES PC # 282, LAUREL, MD, 20707, US

(73) Assignee: Direction Technology Co. Ltd.

(21) Appl. No.: 10/375,360

(22) Filed: Feb. 28, 2003

(51) Int. Cl. A61B 5/0402

(52) U.S. Cl. 600/509

(57) ABSTRACT

A novel mechanism of wireless heartbeat transmitter is composed of a body having an integrated structure, an upper cover, a circuit board, a battery, and a battery cover, which said body integrates the front section of conductible rubber monitoring the heartbeat signal and the shell having its interior accommodating the circuit board into one mechanism so that the front section of said conductible rubber becomes a part of internal mechanism of said shell to reach a superior effect of waterproof and dustproof and form a mechanism of electrical connection or conductible path by directly surface to surface touch of circuit board and conductible rubber.

### BACKGROUND OF THE INVENTION

1. Field of the Present Invention

The present invention relates to a wireless heartbeat transmitter with a specific mechanism of electrical connection by directly touching circuit board and conductible rubber together, and more particularly to a shell mechanism of a wireless heartbeat transmitter which integrates the front section of conductible rubber incorporated into the interior of said shell and become a part of internal mechanism of said shell.

2. Description of Prior Art

The prior wireless heartbeat transmitter basically includes a conductible rubber used as an electrode for monitoring heartbeat signal, and a circuit board having a signal transmitter. While the conductible rubber and the circuit board form an electrical connection, the heartbeat signal monitored by the conductible rubber is wirelessly transmitted by the signal transmitter of the circuit board to a separate but corresponding receiver to reach the goal for wirelessly monitoring an user's heartbeat.

Therefore, the key point of the prior wireless heartbeat transmitter regarding the quality and the accuracy of heartbeat signal is that whether the electrical connection between the conductible rubber and the circuit board is good or not, which means that the structure of electrical connection or conductivity path between the conductible rubber and the circuit board needs to be durable and steady enough to endure the disturbance of vibration and to refrain from dirt, damp, and wearing, and the structure of said conductivity path needs to be simpler to transmit more accurate monitored heartbeat signal.

However, the structure of electrical connection or conductivity path between the conductible rubber and circuit board of prior wireless heartbeat transmitter is too complicated and vulnerable to dirt, damp and wearing, and causes lack of accuracy on the heartbeat signal monitored by its conductible rubber.

Referring to FIG. 1, the structure of electrical connection or conductivity path between the circuit board (1) and the conductible rubber (4), disclosed in U.S. Pat. No. 5,778,880 issued to Chen, is to attach the bottom of the front section of the rubber conductible sheet (4) to the top surface of a metal plate (23) and to install a bent spring sheet (11) at each side of the bottom of the circuit board (1); the rubber conductible sheet (4) and the circuit board (1) becomes connected state by the attachment of the bottom of said spring sheet (11) to the top surface of said metal plate (23). This kind of structure of conductivity path is vulnerable to dirt, damp, and wearing, and the spring sheet (11) is easy to suffer mal-contact caused by vibration, which makes the lack of accuracy of the heartbeat signal monitored by the rubber conductible sheet (4).

### SUMMARY OF THE PRESENT INVENTION

The primary purpose of the present invention is to disclose a novel mechanism of wireless heartbeat transmitter which integrates the front section of conductible rubber monitoring the heartbeat signal into a shell having its interior accommodating the circuit board and makes the front section of said conductible rubber become a part of internal mechanism of said shell to reach a superior effect of waterproof and dustproof and to form a mechanism of electrical connect or conductible path by directly touching the circuit board and the conductible rubber.

Therefore, the wireless heartbeat transmitter of the present invention is durable and steady enough to endure the disturbance of vibration and to refrain from dirt, damp, and wearing; consequently, the heartbeat signal monitored by said conductible rubber is more accurately and wirelessly transmitted through the signal transmitter of said circuit board.

The secondary purpose of the present invention is to disclose a novel mechanism of wireless heartbeat transmitter which is composed of a body having an integrated structure, a upper cover, a circuit board, a battery, and a battery cover, wherein the body is composed of a shell with its interior to accommodate a circuit board, two fixing bands which are integrally molded to both sides of said shell, and two conductible rubbers of which front section of each conductible rubbers become a part of internal mechanism of said shell so that the conductible area at the bottom of said circuit board directly contacts the front section of said conductible rubber; and the portion other than the front section of each conductible rubber is integrally molded and wrapped at the lower side or rear side of fixing band respectively and most bottom of each conductible rubbers are then exposed to the exterior of corresponding fixing bands respectively.

The other purpose of the present invention is to disclose a novel waterproof and dustproof mechanism to wireless heartbeat transmitter, by way of the upper cover with specific structure used to seal the internal room of said shell, so that water or dust are unable to penetrate into said shell of said body due to a multiple-corners turned mechanism formed by the gap between said upper cover and said shell, which completely prevents the interior of said shell of said body from being damped.

### BRIEF DESCRIPTION OF THE DRAWING FIGURES

FIG. 1 is a mechanism of wireless heartbeat transmitter of prior art.

FIG. 2 is a perspective drawing of the wireless heartbeat transmitter of the present invention.

FIG. 3 is an explosion drawing of the wireless heartbeat transmitter of the present invention.

FIG. 4 is an enlarged cross-sectional view along a-a lines of FIG. 3.

FIG. 5 is an enlarged cross-sectional view along b-b lines of FIG. 2.

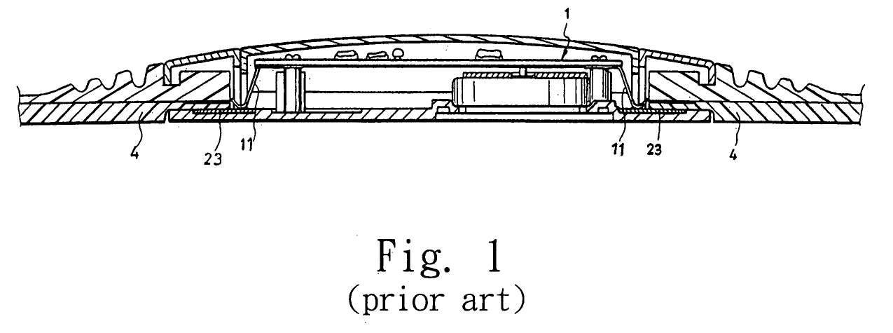

### DETAILED DESCRIPTION OF THE PREFERRED EMBODIMENTS

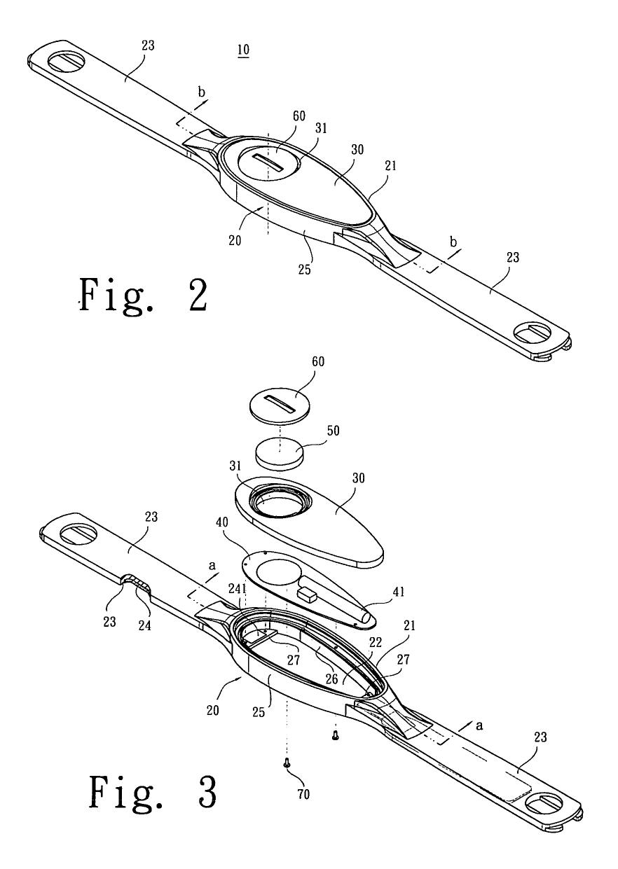

The essential part of the present invention is to provide a body (20) having an integrated structure as shown in FIG. 2 to FIG. 4, which body (20) is installed with an upper cover (30), an circuit board (40), a battery (50), and a battery cover (60) to compose a wireless heartbeat transmitter (10), wherein the electric connection structure and conductivity path structure are especially superior to those of prior art; consequently, not only is the accuracy of heartbeat monitoring signal greatly increased, but also the waterproof effect is much better than that of prior art.

Referring to FIG. 3 and FIG. 4, the body (20) of the present invention is an integrated structure having a shape of watch band, of which the overall structure is composed of a shell (21), two fixing bands (23) which are integrally molded to both sides of said shell (21), and two conductible rubbers (24); wherein said two conductible rubbers (24) are preformed inserts which are first incorporated into the mold during the inject-molding procedure of said body (20), then are inject-molded to manufacture said body (20); said two fixing bands (23) are thus integrally molded to both sides of said shell (21), and the front section (241) of each conductible rubber (24) is formed to said shell (21) as one body; the portion other than the front section (241) of each conductible rubber (24) is integrally molded and wrapped at the lower side or rear side of fixing band (23) respectively so that most bottom of said conductible rubber (24) is exposed to the exterior of fixing band (23) respectively and is used to contact user's skin to monitor the heartbeat signal.

The shell (21) of said body (20) forms a box structure having an internal concave, so that the interior of said shell (21) has an accommodating room (28); in other word, said shell (21) is formed by a base (22) having flat surfaces and by a circumferential side wall which has a relatively higher side rib frame (25) and a relatively lower but thicker locking wall (26) both bulging from the circumference of said base (22) to form a circumferential side wall of said shell (21), wherein said side rib frame (25) forms an external frame of said shell (21) and said locking wall (26) forms an internal wall of said shell (21); moreover, a groove (261) having a rectangular section is made at and around the top of said locking wall (26); therefore, a room formed by said base (22), said side rib frame (25), and said locking wall (26) becomes an accommodating room (28) of said shell (21).

In addition, a couple of platforms (27) bulging from both left and right sides of the bottom of said accommodating room (28) are integrally molded with a front section (241) of a conductible rubber (24), i.e., the front section (241) of said conductible rubber (241) is integrally molded and inlaid into the interior of said shell (21) to form a part structure of said platform (27) with its upper surface forming a major surface portion of said platform (27).

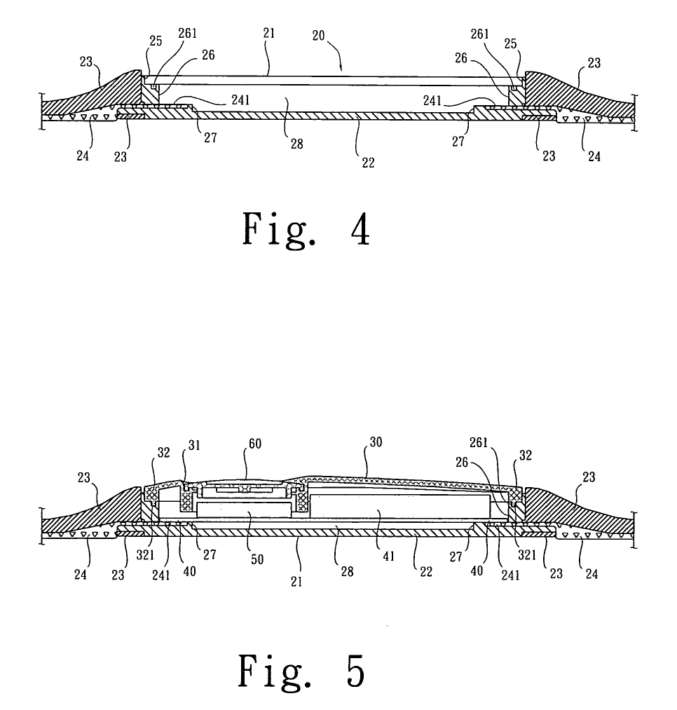

Referring to FIG. 3 and FIG. 5, the surface of the circuit board (40) of the present invention has at least one signal transmitter (41) which wirelessly and accurately transmits the actual heartbeat signal monitored by the conductible rubber (24) at both right and left sides of said body (20) to a receiver which is separated from the wireless heartbeat transmitter (10) of the present invention; therefore, the left and right surfaces at the bottom of said circuit board (40) each forms a conductible area to be electrically connected to each conductible rubber (24).

While the circuit board (40) is installed into the accommodating room (28) of the shell (21) of the body (20), both sides of said circuit board (40) are put respectively upon the surface of each platforms (27) located at the interior of said shell (21), which makes each conductible areas of said circuit board (40) directly connected with each corresponding front section (241) of the conductible rubbers (24) which are integrally molded to the surface of said platforms (27) to form an electrical connection; the circuit board (40) is fixed with screws to make the conductible area at right and left bottom of said circuit board (40) and each corresponding front section (241) of the conductible rubbers (24) form a secure structure provided with an excellent electrical connection adherence and conductivity path to refrain from mal-connection or disturbance of external vibration. Moreover, the body (20) of the present invention is an integral structure, so that the interior of the shell (21), especially at the area around said platform (27) including the front section (241) of the conductible rubber (24), has superior waterproof effect capably to increase the accuracy of the heartbeat signal monitored by conductible rubber (24).

An upper cover (30) is covered to the shell (21) of said body (20) to seal the accommodating room (28) due to achieve waterproof effect. A battery cabin (31) is opened on the surface of the upper cover (30), then a battery (50) used to power source supplied to said circuit board (40) is put on said circuit board (40) through said battery cabin (31); besides, said battery cabin (31) has threads on its cabin wall to joint the battery cover (60) and seal said battery cabin (31).

Referring to FIG. 5, the preferred embodiment of the present invention is further to have the upper cover (30) around its bottom surface provided a loop of barged lip rib (32) with an appropriate thickness, and a protruding rib (321) with a rectangular cross-sectional shape is protruded on the platform of the lip rib (32); while said upper cover (30) is covered to said shell (21) of said body (20), the bottom of said lip rib (32) of said upper cover (30) is completely attached to the top of locking wall (26) at the interior of said shell (21) of said body (20), and the overall side of said lip rib (32) of said upper cover (30) is adjacent to the internal side of said side rib frame (25) of said shell (21); meanwhile, the protruding rib (321) protruded from said platform of said lip rib (32) is completely inserted into the groove (261) at the top of said locking wall (26); therefore, after the upper cover (30) is locked with screws, water or dust is unable to penetrate into said shell (21) of said body (20) due to an invented multiple-comers turned mechanism formed by the gaps between said protruding rib (321) of said upper cover (30) and said groove (261) of said shell (21), which completely prevents the interior of said shell (21) of said body (20) from being moisturized and obtains a superior waterproof and dustproof effect.

To summarize, the wireless heartbeat transmitter (10) of the present invention not only integrates the front section (241) of conductible rubber (24) monitoring the heartbeat signal and the shell (21) having its interior accommodating the circuit board (40) into one mechanism, but also makes the front section (241) of said conductible rubber (24) become a part of internal mechanism of said shell (21), which reaches an superior effect of waterproof and dustproof and forms a mechanism of electrical connect or conductible path by directly touch of circuit board (40) and conductible rubber (24).

Therefore, the wireless heartbeat transmitter (10) disclosed by the present invention is durable and steady enough to endure the disturbance of vibration and to refrain from dirt, damp, and wearing; consequently, the heartbeat signal monitored by said conductible rubber (24) is accurately and wirelessly transmitted through the signal transmitter (41) of said circuit board (40).

What is claimed is:

1. A mechanism of wireless heartbeat transmitter, which comprises a body formed as an integrated structure having a shape of watch band, an upper cover, and a circuit board having a signal transmitter, wherein said body is integrally composed of a shell having its interior to accommodate said circuit board inside, two fixing bands integrally molded to both sides of said shell, and two conductible rubbers of which front section are respectively formed a portion of internal structure of the shell of said body and the other part of each conductible rubbers are integrally molded and wrapped at the rear side of the corresponding fixing band respectively; said circuit board is securely installed inside the shell of said body to have its conductible area at left and right bottom sides directly touched onto the front section of each corresponding conductible rubbers as an electrical connection; and said upper cover is covered to the shell of said body for sealing.

2. The mechanism of wireless heartbeat transmitter as defined in claim 1, wherein the shell of said body forms a box structure having an internal concave, said shell is formed by a base having flat surfaces and by a circumferential side wall which has a relatively higher side rib frame and a relatively lower locking wall both bulging from the circumference of said base to form a circumferential side wall of said shell, said side rib frame forms an external frame of said shell and said locking wall forms an internal wall of said shell; which front section of each conductible rubbers are integrally molded and incorporated into the interior of said shell respectively to form a part of the internal structure of said shell; and said upper cover is covered to the top surface of said locking wall of said shell.

3. The mechanism of wireless heartbeat transmitter as defined in claim 2, wherein the top surface of said locking wall has a loop of groove circling the top surface of the overall locking wall; and said upper cover bulges a loop of protruding rib corresponding to the groove of said locking wall and inserted into said groove when assembly.

4. The mechanism of wireless heartbeat transmitter as defined in claim 3, wherein the groove at the top surface of said locking wall of said shell has a rectangular cross-sectional shape.

5. The mechanism of wireless heartbeat transmitter as defined in claim 1, wherein said upper cover has a battery cabin.

6. The mechanism of wireless heartbeat transmitter as defined in claim 5, wherein the shell of said body forms a box structure having an internal concave, said shell is formed by a base having flat surfaces and by a circumferential side wall which has a relatively higher side rib frame and a relatively lower locking wall both bulging from the circumference of said base to form a circumferential side wall of said shell, said side rib frame forms an external frame of said shell and said locking wall forms an internal wall of said shell; which front section of each conductible rubbers are integrally molded and incorporated into the interior of said shell respectively to form a part of the internal structure of said shell; and said upper cover is covered to the top surface of said locking wall of said shell.

7. The mechanism of wireless heartbeat transmitter as defined in claim 6, wherein the top surface of said locking wall has a loop of groove circling the top surface of the overall locking wall; and said upper cover bulges a loop of protruding rib corresponding to the groove of said locking wall and inserted into said groove when assembly.

8. The mechanism of wireless heartbeat transmitter as defined in claim 7, wherein the groove at the top surface of said locking wall of said shell has a rectangular cross-sectional shape.
